(19) United States
(12) Patent Application Publication
(10) Pub. No.: US 2024/0115530 A1
(43) Pub. Date: Apr. 11, 2024

(54) ADMINISTRATION OF BAIBA TO INCREASE BENEFIT OF LOSING WEIGHT OF INTERMITTENT FASTING

(71) Applicant: NANJING NUTRABUILDING BIO-TECH CO., LTD., Nanjing (CN)

(72) Inventors: Ronghua YI, Nanjing (CN); Kylin LIAO, Nanjing (CN)

(21) Appl. No.: 18/392,313

(22) Filed: Dec. 21, 2023

Related U.S. Application Data
(63) Continuation of application No. PCT/CN2022/100011, filed on Jun. 21, 2022.

(30) Foreign Application Priority Data
Jun. 22, 2021 (WO) PCT/CN2021/101529

(51) Int. Cl. A61K 31/197; A61P 3/04

(52) U.S. Cl. CPC A61K 31/197; A61P 3/04

(57) ABSTRACT

The present invention provides methods for providing a mammal with a benefit associated with every-other-day fasting (EODF), or increasing a benefit of losing weight of a mammal treated with every-other-day fasting (EODF), comprising administrating to the mammal a therapeutically effective amount of β-aminoisobutyric acid (BAIBA), an analog or derivative thereof, or a pharmaceutically acceptable salt, acid, ester, polymer, analog or derivative thereof. The benefit may include reducing a body fat percentage of the mammal, reducing the weight of the mammal, lowering a blood glucose of the mammal, decreasing a blood triglyceride level of the mammal, decreasing a blood total cholesterol level of the mammal, decreasing a blood low-density lipoprotein level of the mammal, decreasing a blood very low-density lipoprotein level of the mammal, improving leptin resistance of the mammal, and/or improving insulin resistance of the mammal.

### CROSS-REFERENCE TO RELATED APPLICATION

This application is a continuation application of International Patent Application No. PCT/CN2022/100011, filed on Jun. 21, 2022, which claims the priority of the International Application No. PCT/CN2021/101529, filed on Jun. 22, 2021, the contents of all of which are incorporated herein by reference in their entirety.

### FIELD OF THE INVENTION

This invention generally relates to the field of weight loss or control, and more specifically relates to compositions and methods for providing or increasing a benefit of losing weight (e.g., associated with every-other-day fasting) of a mammal in need thereof, with administration of a therapeutically effective amount of BAIBA, an analog or derivative thereof, or a pharmaceutically acceptable salt, acid, ester, analog or derivative thereof, as an active ingredient.

### BACKGROUND

Beta-aminoisobutyric acid (BAIBA) is a natural catabolite of thymine, and a non-protein amino acid secreted by skeletal muscles upon regular exercise via peroxisome proliferator-activated receptor gamma coactivator 1-alpha. There are two enantiomers of BAIBA in biological systems: D-BAIBA (R-BAIBA) and L-BAIBA (S-BAIBA). D-BAIBA is produced in cytosol as an intermediate product of thymine degradation, while L-BAIBA comes from mitochondrial reactions of L-valine catabolism.

Obesity is a common disease nowadays, associated with decreased life span and numerous medical problems with economic development, which may be a risk factor for cancers, heart disease, and type 2 diabetes. Intermittent fasting is a beneficial dietary treatment for obesity through lowering fasted insulin levels, improving glucose tolerance, and lowering blood cholesterol. As one of the main intermittent fasting strategies, every-other-day fasting (EODF) is a simple and easily implemented strategy and has been established to be tolerable in human trials. As such, EODF is a common option for combating metabolic disease. Nevertheless, recent studies in mice based on proteomics showed an increase in mitochondrial protein content in subcutaneous white adipose tissue (scWAT) and visceral WAT (vWAT) depots after EODF, and this effect is correlated with increased fatty acid synthesis enzymes in both WAT depots but not in brown adipose tissue. Strikingly, it showed that EODF treatment downregulates lipolysis specifically in vWAT, indicating the preservation of the visceral lipid store during EODF. In other words, the preservation of the visceral lipid store during EODF, namely lipolysis resistance, may compromise the effect of losing weight through EODF.

To overcome the drawbacks of conventional weight-loss methods, it is desired to provide improved methods and compositions to increase fat mobilization during EODF, thereby increasing the benefit of losing weight of EODF.

### SUMMARY

This summary is provided to introduce a selection of concepts in a simplified form that is further described below in the Detailed Description. This summary is not intended to identify key features or essential features of the claimed subject matter, nor is it intended to be used to limit the scope of the claimed subject matter.

The present invention generally relates to compositions and methods for providing or increasing a benefit of losing weight of a mammal—particularly a mammal treated with every-other-day fasting (EODF), comprising administrating to the mammal (e.g., engaged in an EODF regimen) a therapeutically effective amount of β-aminoisobutyric acid (BAIBA), an analog or derivative thereof, or a pharmaceutically acceptable salt, acid, ester, polymer, analog or derivative thereof.

BAIBA was found to prevent obesity and related metabolic disorders in different murine models. As a novel endogenous protective myokine, BAIBA may induce transition of white adipose tissue to a “beige” phenotype (the differentiation of resident progenitor cells in white adipose tissue into morphologically and physiologically distinct brown-like adipocytes), resulting in mitochondrial fatty acids oxidation (FAO), body weight reduction, and improvement of diet-induced insulin resistance in mice.

According to this invention, it was surprisingly found that administration of BAIBA can particularly increase fat mobilization during EODF in obese mice, thereby increasing the benefit of losing weight of EODF.

One aspect of this invention relates to a method for providing a mammal with a benefit associated with every-other-day fasting (EODF), or increasing a benefit of losing weight of a mammal treated with every-other-day fasting (EODF), comprising administrating to the mammal (e.g., engaged in an EODF regimen) a therapeutically effective amount of β-aminoisobutyric acid (BAIBA), an analog or derivative thereof, or a pharmaceutically acceptable salt, acid, ester, polymer, analog or derivative thereof.

In some embodiments, the benefit comprises reducing a body fat percentage of the mammal, reducing the weight of the mammal, lowering a blood glucose of the mammal, decreasing a blood triglyceride level of the mammal, decreasing a blood total cholesterol level of the mammal, decreasing a blood low-density lipoprotein level of the mammal, decreasing a blood very low-density lipoprotein level of the mammal, improving leptin resistance of the mammal, and/or improving insulin resistance of the mammal.

In some embodiments, the benefit of losing weight of the EODF regimen is greater with administration of BAIBA than the same EODF regimen without administration of the BAIBA.

In some embodiments, BAIBA comprises L-BAIBA, D-BAIBA, or a combination thereof.

In some embodiments, BAIBA to be administrated comprises L-β-aminoisobutyric acid (L-BAIBA) present in an amount ranging from 1% to 100% of the total amount of BAIBA. For instance, BAIBA to be administrated may comprise L-β-aminoisobutyric acid (L-BAIBA) present in an amount of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% of the total amount of BAIBA.

In some embodiments, BAIBA is administrated orally, by intravenous injection, by intramuscular injection, intraperitoneally or sublingually.

In some embodiments, BAIBA is administrated in an ingestible composition. The ingestible composition may be selected from the group consisting of a bioceutical composition, a dietary supplement, a medicated feed, a nutraceutical composition, and a pharmaceutical composition.

In some preferred embodiments, the ingestible composition is a pharmaceutical composition comprising L-BAIBA as an active pharmaceutical ingredient.

In some embodiments, BAIBA is administrated in a form of aqueous solutions, aqueous suspensions, capsules, drops, granules, liquids, powders, syrups, tablets, functionalized foods, beverages, toothpastes, and sublingual articles.

In some embodiments, the mammal is a human or animal (e.g., pet or cattle). In some embodiments, the mammal suffers from a disorder selected from the group consisting of pre-obesity, obesity, and hyperglycemia.

In some embodiments, BAIBA is administrated once per day repeatedly for a period of between about one week and about twelve weeks (e.g., between about eight weeks and about twelve weeks).

In some embodiments, BAIBA is administered to the mammal with a dose ranging from about 1 mg/kg/day to about 200 mg/kg/day (e.g., from about 5 mg/kg/day to about 200 mg/kg/day).

In some embodiments, L-BAIBA is administered to the mammal in an amount ranging from about 1 mg/kg/day to about 200 mg/kg/day (e.g., from about 5 mg/kg/day to about 200 mg/kg/day).

Another aspect of the present invention provides a use of BAIBA for preparing a composition for providing or increasing a benefit of losing weight of a mammal in need thereof, wherein the composition comprises a therapeutically effective amount of BAIBA, an analog or derivative thereof, or a pharmaceutically acceptable salt, acid, ester, analog or derivative thereof, as an active ingredient.

In some embodiments, the mammal is treated with every-other-day fasting (EODF).

In some embodiments, the benefit comprises reducing a body fat percentage of the mammal, reducing the weight of the mammal, lowering a blood glucose of the mammal, decreasing a blood triglyceride level of the mammal, decreasing a blood total cholesterol level of the mammal, decreasing a blood low-density lipoprotein level of the mammal, decreasing a blood very low-density lipoprotein level of the mammal, improving leptin resistance of the mammal, and/or improving insulin resistance of the mammal.

In some embodiments, the benefit of losing weight of the EODF regimen is greater with administration of BAIBA than the same EODF regimen without administration of the BAIBA.

In some embodiments, the mammal is a human or animal. (e.g., pet or cattle). In some embodiments, the mammal suffers from a disorder selected from the group consisting of pre-obesity, obesity, and hyperglycemia.

In some embodiments, BAIBA comprises L-BAIBA, D-BAIBA, or a combination thereof.

In some embodiments, the composition comprises L-β-aminoisobutyric acid (L-BAIBA) present in an amount ranging from 1% to 100% of the total amount of BAIBA. For instance, the composition may comprise L-β-aminoisobutyric acid (L-BAIBA) present in an amount of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% of the total amount of BAIBA.

In some embodiments, the composition is prepared in a form of aqueous solutions, aqueous suspensions, capsules, drops, granules, liquids, powders, syrups, tablets, functionalized foods, beverages, toothpastes, and sublingual articles.

In some embodiments, the composition is a pharmaceutical composition comprising L-BAIBA as an active pharmaceutical ingredient.

In some embodiments, the composition is administrated once per day repeatedly for a period of between about one week and about twelve weeks (e.g., between about eight weeks and about twelve weeks).

In some embodiments, the composition comprises a dose of BAIBA, wherein the dose of BAIBA ranges from about 1 mg/kg/day to about 200 mg/kg/day (e.g., from about 5 mg/kg/day to about 200 mg/kg/day).

Still, in some embodiments, the composition comprises a dose of L-BAIBA, wherein the dose of L-BAIBA ranges from about 1 mg/kg/day to about 200 mg/kg/day (e.g., from about 5 mg/kg/day to about 200 mg/kg/day).

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

### BRIEF DESCRIPTION OF THE DRAWINGS

FIG. 1 is the average body weight of mice in all test groups.

FIG. 2a-2d are the results of TG, TCHO, LDL and VLDL in all test groups, respectively.

FIG. 3a-3b are the serum levels of insulin and leptin in all test groups.

FIG. 4 is the changes of blood glucose at different time points in all test groups.

### DETAILED DESCRIPTION

Reference will now be made in detail to the preferred embodiments of the invention, examples of which are further illustrated. While the invention will be described in conjunction with the preferred embodiments, it will be understood that they are not intended to limit the invention to these embodiments. On the contrary, the invention is intended to cover alternatives, modifications, and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

Generally speaking, various embodiments of the present invention provide for providing a mammal with a benefit associated with every-other-day fasting (EODF), or increasing a benefit of losing weight of a mammal treated with every-other-day fasting (EODF), comprising administrating to the mammal (e.g., engaged in an EODF regimen) an effective amount of β-aminoisobutyric acid (BAIBA), an analog or derivative thereof, or a pharmaceutically acceptable salt, acid, ester, polymer, analog or derivative thereof. It was surprisingly found that administration of BAIBA can particularly increase fat mobilization during EODF in obesity mice, thereby increasing the benefit of losing weight of EODF. For instance, the benefit may include reducing a body fat percentage of the mammal, reducing the weight of the mammal, lowering a blood glucose of the mammal, decreasing a blood triglyceride level of the mammal, decreasing a blood total cholesterol level of the mammal, decreasing a blood low-density lipoprotein level of the mammal, decreasing a blood very low-density lipoprotein level of the mammal, improving leptin resistance of the mammal, and/or improving insulin resistance of the mammal. The mammal may from a disorder selected from the group consisting of pre-obesity, obesity, and hyperglycemia. Moreover, BAIBA comprises L-BAIBA, D-BAIBA, or a combination thereof. For instance, L-BAIBA may present in an amount ranging from 1% to 100% of the total amount of BAIBA—e.g., of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% of the total amount of BAIBA. In some embodiments, BAIBA (e.g., L-BAIBA) is administered (e.g., as an active ingredient) to the mammal with a dose ranging from about 1 mg/kg/day to about 200 mg/kg/day (e.g., from about 5 mg/kg/day to about 200 mg/kg/day). BAIBA may also be administrated once per day repeatedly for a period of between about one week and about twelve weeks (e.g., between about eight weeks and about twelve weeks).

The following examples are illustrative of select embodiments of the present invention and are not meant to limit the scope of the invention.

### Example 1: Body Weight Reduction in Mice

50 C57BL/6 male mice aged about 8 weeks, are randomly assigned to five study groups (GA, GB1, GB2, GB3, GB4) of ten mice each (two cages, 1 cage, n=5 per condition). Each animal's body weight is measured by a weight scale and recorded.

The total experimental time is 19 weeks, including two periods: an eleven-week induction period and a subsequent eight-week treatment period.

Each study group consisted of ten animals, and treatment details of the individual study groups are given below:
Group A (GA, NC): Normal mice, maintained on normal diet for 11 weeks, receiving neither EODF nor L-BAIBA treatment.
Group B1 (GB1, HFD): Obese mice, maintained on high fat diet (HFD) for 11 weeks, receiving neither EODF nor L-BAIBA treatment.
Group B2 (GB2, HFD+EODF): Obese mice, maintained on HFD for 11 weeks, receiving only EODF and no L-BAIBA treatment.
Group B3 (GB3, HFD+BAIBA): Obese mice, maintained on HFD for 11 weeks, receiving only L-BAIBA treatment and no EODF.
Group B4 (GB4, HFD+EODF+BAIBA): Obese mice, maintained on HFD for 11 weeks, receiving L-BAIBA treatment and EODF combination.

L-BAIBA was administered daily via per oral route according to a level of 150 mg/kg body weight. Animals not receiving L-BAIBA were given orally with ultrapure water.

Animals were maintained in a pathogen free condition under a strict 12 h light/dark cycle (0600/1800 h) with 12-15 cycles/hour of air change.

Animals were provided with access to respective feed and water ad libitum as per experimental conditions. Animals were housed under controlled laboratory conditions of temperature and humidity at 23±2° C. and 50±10% RH, respectively.

Intermittent fasting model: HFD fed male mice in groups at 8 weeks of age were randomly assigned into either ad libitum, or every-other-day-fasting (EODF) groups on a per cage basis. All cage bedding was changed to paper bedding for the duration of the model. Mice in the EODF group had total deprivation of food and ad libitum access to water from 1200 h-1200 h on alternate days with ad libitum food and water access. EODF cages were changed upon induction of fasting. Ad libitum control mice cages were changed every other day with fresh food provided.

Each mouse's body weight is measured weekly during the study at 1200 h. FIG. 1 is the average body weight of mice in all test groups (Body weight were recorded from the beginning of the experiment). As shown, over the course of the study, HFD fed mice in groups receiving L-BAIBA supplementation with or without EODF undergo a greater reduction in body weight, relative to mice subjected to the same HFD with and without EODF regimen but not receiving L-BAIBA supplementation, indicating that supplementing L-BAIBA can achieve or even exceed the beneficial effects of EODF on weight loss. This effect persists for as long as the test protocol is continued.

### Example 2: Improvement in Biochemistry

50 C57BL/6 male mice aged about 8 weeks, are randomly assigned to five study groups (GA, GB1, GB2, GB3, GB4) of ten mice each (two cages, 1 cage, n=5 per condition).

The same procedures were conducted as above-discussed Example 1.

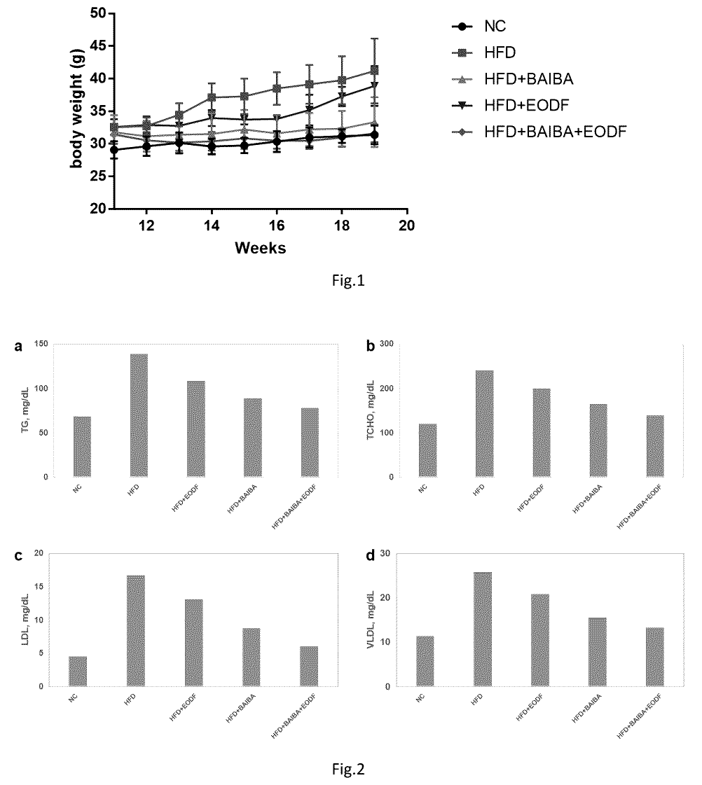

At the end of the study, blood was collected from all test groups and a various clinical parameter were determined: triglyceride (TG), total cholesterol (TCHO), low-density lipoprotein (LDL) and very low-density lipoprotein (VLDL). FIG. 2a-2d are the results of TG, TCHO, LDL and VLDL in all test groups, respectively. In FIG. 2a-2d, it is observed that mice in groups receiving L-BAIBA supplementation have lower levels of TG, TCHO, LDL and VLDL relative to mice subjected to the same HFD with and without EODF regimen but not receiving L-BAIBA supplementation, indicating that supplementing L-BAIBA can achieve or even exceed the beneficial effects of EODF on improving biomarkers related to lipid mobilization than EODF only, and EODF regimen combines with supplementing L-BAIBA can have greater effect on weight loss than EODF only. This effect persists for as long as the test protocol is continued.

### Example 3: Improvement Insulin Resistance and Leptin Resistance

50 C57BL/6 male mice aged about 8 weeks, are randomly assigned to five study groups (GA, GB1, GB2, GB3, GB4) of ten mice each (two cages, 1 cage, n=5 per condition).

The same procedures were conducted as above-discussed Example 1.

Serum levels of insulin and leptin were determined at the end of the treatment period by standard ELISA methods. Since one key feature of obesity is the development of insulin and leptin resistance resulting in an elevation of the circulating levels of these two hormones as a consequence of compensatory physiological responses to insulin and leptin insensitivity, thus leptin is one of the major adipocytokines associated with maintaining glucose homeostasis.

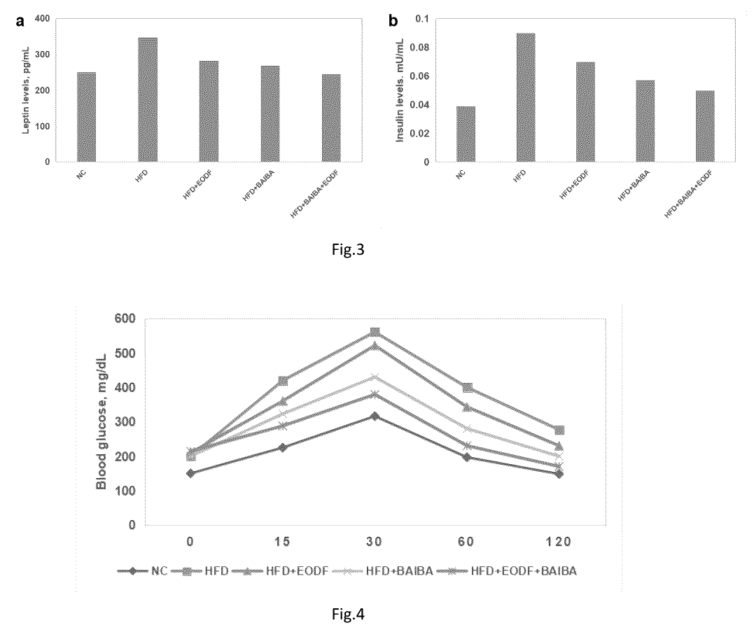

FIG. 3a-3b are the serum levels of insulin and leptin in all test groups. In FIG. 3, it is observed that mice in groups receiving L-BAIBA supplementation have improved leptin resistance and insulin resistance relative to mice subjected to the same HFD with and without EODF regimen but not receiving L-BAIBA supplementation, indicating that supplementing L-BAIBA can achieve or even exceed the beneficial effects of EODF on improving leptin resistance and insulin resistance of obese mice than EODF only, and EODF regimen combines with supplementing L-BAIBA can have a greater effect on improving leptin resistance and insulin resistance of obese mice than EODF only. This effect persists for as long as the test protocol is continued.

### Example 4: Blood Glucose Reduction in Mice

50 C57BL/6 male mice aged about 16 weeks, are randomly assigned to five study groups (GA, GB1, GB2, GB3, GB4) of ten mice each (two cages, 1 cage, n=5 per condition).

The same procedures were conducted as above-discussed Example 1.

Glucose levels in blood were measured weekly (week 12-19) on the starting day of every week before the day's EODF and L-BAIBA dosing activities. Towards the end of the final week of the study, an oral glucose tolerance test (OGTT) was performed on day after the treatment period for all animals (NC and HFD groups).

FIG. 4 is the changes of blood glucose at different time points in all test groups. As indicated, over the course of the study, mice in groups receiving L-BAIBA supplementation undergo a greater reduction in blood glucose level, maintained the blood glucose level of obese mice at a stable level and near to the level of normal mice (GA, NC) throughout the experimental period, and showed better glucose tolerance relative to mice subjected to the same HFD with and without EODF regimen but not receiving L-BAIBA supplementation, indicating that supplementing L-BAIBA can achieve or even exceed the beneficial effects of EODF on reducing the blood glucose levels, improving the glucose tolerance of obese mice than EODF only, and EODF regimen combines with supplementing L-BAIBA can have greater effect on reducing the blood glucose levels, improving the glucose tolerance of obese mice than EODF only. This effect persists for as long as the test protocol is continued.

Although specific embodiments and examples of this invention have been illustrated herein, it will be appreciated by those skilled in the art that any modifications and variations can be made without departing from the spirit of the invention. The examples and illustrations above are not intended to limit the scope of this invention. Any combination of embodiments of this invention, along with any obvious their extension or analogs, are within the scope of this invention. Further, it is intended that this invention encompass any arrangement, which is calculated to achieve that same purpose, and all such variations and modifications as fall within the scope of the appended claims.

All the features disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example of a generic series of equivalent or similar features.

### Other Embodiments

It is to be understood that while the invention has been described in conjunction with the detailed description thereof and accompanying figures, the foregoing description and accompanying figures are only intended to illustrate, and not limit the scope of the invention, which is defined by the scope of the appended claims. Other aspects, advantages, and modifications are within the scope of the following claims. All publications referenced herein are incorporated by reference in their entireties.

What is claimed is:

1. A method for providing a mammal with a benefit associated with every-other-day fasting (EODF), or increasing a benefit of losing weight of a mammal treated with every-other-day fasting (EODF), comprising administrating to the mammal a therapeutically effective amount of β-aminoisobutyric acid (BAIBA), an analog or derivative thereof, or a pharmaceutically acceptable salt, acid, ester, polymer, analog or derivative thereof.

2. The method of claim 1, wherein the benefit comprises reducing a body fat percentage of the mammal, reducing the weight of the mammal, lowering a blood glucose of the mammal, decreasing a blood triglyceride level of the mammal, decreasing a blood total cholesterol level of the mammal, decreasing a blood low-density lipoprotein level of the mammal, decreasing a blood very low-density lipoprotein level of the mammal, improving leptin resistance of the mammal, and/or improving insulin resistance of the mammal.

3. The method of claim 1, wherein the mammal is engaged in an EODF regimen, and the benefit of losing weight of the EODF regimen is greater with administration of BAIBA than the same EODF regimen without administration of the BAIBA.

4. The method of claim 1, wherein BAIBA comprises L-BAIBA, D-BAIBA, or a combination thereof.

5. The method of claim 1, wherein BAIBA to be administrated comprises L-β-aminoisobutyric acid (L-BAIBA) present in an amount ranging from 1% to 100% of the total amount of BAIBA.

6. The method of claim 1, wherein BAIBA to be administrated comprises L-β-aminoisobutyric acid (L-BAIBA) present in an amount of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% of the total amount of BAIBA.

7. The method of claim 1, wherein BAIBA is administrated orally, by intravenous injection, by intramuscular injection, intraperitoneally or sublingually.

8. The method of claim 7, wherein BAIBA is administrated in an ingestible composition, wherein the ingestible composition is selected from the group consisting of a bioceutical composition, a dietary supplement, a medicated feed, a nutraceutical composition, and a pharmaceutical composition.

9. The method of claim 1, wherein BAIBA is administrated in a form of aqueous solutions, aqueous suspensions, capsules, drops, granules, liquids, powders, syrups, tablets, functionalized foods, beverages, toothpastes, and sublingual articles.

10. The method of claim 1, wherein the mammal suffers from a disorder selected from the group consisting of pre-obesity, obesity, and hyperglycemia.

11. The method of claim 1, wherein BAIBA is administered to the mammal with a dose ranging from about 1 mg/kg/day to about 200 mg/kg/day.

12. The method of claim 1, wherein L-BAIBA is administered to the mammal in an amount ranging from about 1 mg/kg/day to about 200 mg/kg/day.

13. Use of BAIBA for preparing a composition for providing or increasing a benefit of losing weight of a mammal in need thereof, wherein the composition comprises a therapeutically effective amount of BAIBA, an analog or derivative thereof, or a pharmaceutically acceptable salt, acid, ester, analog or derivative thereof, as an active ingredient.

14. The use of claim 13, wherein the mammal is treated with every-other-day fasting (EODF).

15. The use of claim 13, wherein the benefit comprises reducing a body fat percentage of the mammal, reducing the weight of the mammal, lowering a blood glucose of the mammal, decreasing a blood triglyceride level of the mammal, decreasing a blood total cholesterol level of the mammal, decreasing a blood low-density lipoprotein level of the mammal, decreasing a blood very low-density lipoprotein level of the mammal, improving leptin resistance of the mammal, and/or improving insulin resistance of the mammal.

16. The use of claim 14, wherein the benefit of losing weight of the EODF regimen is greater with administration of BAIBA than the same EODF regimen without administration of the BAIBA.

17. The use of claim 13, wherein BAIBA comprises L-BAIBA, D-BAIBA, or a combination thereof.

18. The use of claim 17, wherein the composition comprises L-β-aminoisobutyric acid (L-BAIBA) present in an amount ranging from 1% to 100% of the total amount of BAIBA.

19. The use of claim 13, wherein the composition is prepared in a form of aqueous solutions, aqueous suspensions, capsules, drops, granules, liquids, powders, syrups, tablets, functionalized foods, beverages, toothpastes, and sublingual articles.

20. The use of claim 13, wherein the composition comprises a dose of BAIBA, wherein the dose of BAIBA ranges from about 1 mg/kg/day to about 200 mg/kg/day.
